SEQUENCE LISTING (8 sequences)

SEQ ID NO: 1 - DNA, 3394 bp, Artificial Sequence
  High copy plasmid with Lactococcus P44 promoter
taacaattgtaacccatccaggagacgggacgatagcaattttttcaataagtagacaaagtagagaataatttaataaaaaactgaaaaaatcacaggtaaactcttgttttactgtgattttatgttaaaataattaatgagtgtaattgtatataaaattatctgtacacttacctaatttattgaaaaaaatatgaatcgtgatgtgtgagggaaaggaggcgcttttatggccaaaccatgggtcctgcaggctcgcggtaccattagttctagagagctcaagctttctttgaaccaaaattagaaaaccaaggcttgaaacgttcaattgaaatggcaattaaacaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaagcgagaataaggaagataaatcccataagggcgggagcagaatgtccgagactaatgtaaatttgtccaccaattaaaggaccgataacgcgagcttctcgagtgcatattttcggcaatcttctcaatgagatgctcttcagcatgttcaatgatgtcgattttttattaaaacgtctcaaaatcgtttctgagacgttttagcgtttatttcgtttagttatcggcataatcgttaaaacaggcgttatcgtagcgtaaaagcccttgagcgtagcgtgctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttattgttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaatatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgacggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccttggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcactaacctgccccgttagttgaagaaggtttttatattacagctccaagatc

SEQ ID NO: 2 - DNA, 226 bp, Artificial Sequence
  DNA sequence of putative anti-CRISPR protein found in 100E-D1 with overlap to pNZ44
accatgggtcctgcaggctcgcggtaccattagttatgaaatataaagtaattacatattttgacaacatggaagatgatgtagaagtatttgacagaaaggatgaagctattaacagattgcatcatctacgaggtgttaaatatagaaattcaagattatataaagtagaaatggttgaggtggaatagagagctcaagctttctttgaaccaaaattagaaaa

SEQ ID NO: 3 - DNA, 226 bp, Artificial Sequence
  DNA sequence of putative anti-CRISPR protein found in 200F-D77 with overlap to pNZ44
accatgggtcctgcaggctcgcggtaccattagttatgagatataaagttattgtttattacgacaatatggaagacgatgtagaaatttatgagaataaggatgaagctatcaacagattgcatcatctacgaggtgttaaatatagaaattcaagattatataaagtagaaatggttgaggtggaataaagagctcaagctttctttgaaccaaaattagaaaa

SEQ ID NO: 4 - DNA, 406 bp, Artificial Sequence
  DNA sequence of putative anti-CRISPR protein found in 300F with overlap to pNZ44
accatgggtcctgcaggctcgcggtaccattagttatggaagaattagaacaagcgtttgaaaatcttgatgattggtatctatcaagcatgaaagacagtgcttacaaggattttgggaaatacgaaattcgcttatcaaatcattcggcagacaataaatatcatgacctagaaaatggtcgcttaatcgttaatgttaaagctagtaaattgaacttcgttgatatcatcgagaataaaatctataaaatcattgagaaaattgaaactctcgatttagacaagtacagatttattaacgctactaaaatggaaaacgatatcaaatgttattacaagggatttaagacaaagaaggatgtgatctaaagagctcaagctttctttgaaccaaaattagaaaa

SEQ ID NO: 5 - DNA, 493 bp, Artificial Sequence
  DNA sequence of anti-CRISPR protein found by Hynes et al., 2017 with overlap to pNZ44
accatgggtcctgcaggctcgcggtaccattagttatggcatacggaaaaagtagatataactcatatagaaagcgcagttttaacagaagtaataagcaacgtagagaatacgcacaagaaatggatagattagagaaagctttcgaaaatcttgacggatggtatctatctagcatgaaagacagtgcttacaaggattttgggaaatacgaaattcgcttatcaaatcattcggcagacaacaaatatcacgacttagaaaacggtcgtttaattgttaatattaaagctagtaaattgaatttcgttgatatcatcgagaataagcttgataaaataatcgagaagattgataagcttgatttagataagtaccgattcatcaatgcgaccaacctagagcatgatatcaaatgctattacaaggggtttaaaacgaaaaaggaggtaatctaaagagctcaagctttctttgaaccaaaattagaaaa

SEQ ID NO: 6 - DNA, 622 bp, Artificial Sequence
  DNA sequence of anti-CRISPR protein found by Hynes et al., 2018, with overlap to pNZ44
accatgggtcctgcaggctcgcggtaccattagttatgaaaataaatgacgacatcaaagagttaattttagaatatatgagccgttacttcaaattcgagaacgacttttataaactgccaggcatcaagttcactgatgcaaattggcagaagttcaaaaatggaggcactgacattgagaagatgggggcggcacgagtaaacgccatgctcgactgcctattcgacgatttcgagcttgctatgattggcaaggctcaaactaattattacaatgataattcactaaagatgaacatgccattttacacttactatgacatgttcaaaaagcagcaacttctaaaatggcttaaaaataaccgtgatgatgtcatcggcggaactggtaggatgtacacagcaagtggtaattacattgctaacgcttatttagaggtggcattagaatcgagctcgcttggtagtggctcttacatgcttcaaatgaggtttaaagactattcaaaaggtcaagaacctattccgtcaggtcgtcagaatcgacttgaatggattgaaaacaatctcgaaaacattcgataaagagctcaagctttctttgaaccaaaattagaaaa

SEQ ID NO: 7 - DNA, 19 bp, Artificial Sequence
  forward primer on backbone pNZ44 vector
accatgggtcctgcaggct

SEQ ID NO: 8 - DNA, 35 bp, Artificial Sequence
  reverse primer on backbone pNZ44 vector
ttttctaattttggttcaaagaaagcttgagctct

(19) United States
(12) Patent Application Publication
(10) Pub. No.: US 2022/0064588 A1
(43) Pub. Date: Mar. 3, 2022

(54) PHAGE RESISTANT LACTIC ACID BACTERIA

(71) Applicant: DSM IP ASSETS B.V., Heerlen (NL)

(72) Inventors: Paulus DE WAAL, Echt (NL); Laurens Leendert HANEMAAIJER, Echt (NL)

(21) Appl. No.: 17/414,839

(22) PCT Filed: Dec. 17, 2019
(86) PCT No.: PCT/EP2019/085483

§ 371 Date: Jun. 16, 2021

(30) Foreign Application Priority Data
Dec. 18, 2018 (EP) 18213374.4

(51) Int. Cl. C12N 1/20; C12N 15/01; A23C 19/032; A23C 9/123

(52) U.S. Cl. CPC C12N 1/205; C12N 15/01; C12R 2001/46; A23C 9/1238; A23C 19/0323

(57) ABSTRACT

The present invention relates to a method for obtaining a bacteriophage insensitive mutant of a lactic acid bacterium parent strain suitable for food and feed fermentation. Further, the present invention relates to a method for the construction of a bacteriophage insensitive lactic acid bacterium whereby the phage resistance is conferred by a mechanism other than CRISPR. A preferred lactic acid bacterium is Streptococcus thermophilus.

### FIELD

The present invention relates to a method for obtaining a bacteriophage insensitive mutant of a lactic acid bacterium parent strain suitable for food and feed fermentation. Further, the present invention relates to a method for the construction of a bacteriophage insensitive lactic acid bacterium whereby the phage resistance is conferred by a mechanism other than CRISPR. A preferred lactic acid bacterium is Streptococcus thermophilus.

### BACKGROUND

Streptococcus thermophilus is a Gram-positive thermophilic bacterium used globally as a starter culture in dairy fermentations and is widely employed for the production of cheese and yoghurt products. Despite its usefulness in starter cultures, S. thermophilus (as well as other lactic acid bacteria) remain(s) highly susceptible to (bacterio)phage predation which can lead to substandard or failed fermentations and considerable economic losses. Evidenced by these potentially considerable costs, there is a clear advantage to selecting robust starters which are less susceptible to phage attack and yet retain favourable growth and production characteristics. Combined with effective hygiene and sanitation in industrial fermentation plants, unrelated robust starters used in rotation have the potential to reduce the incidence of phage fermentation disruption.

Phages of S. thermophilus are, despite their narrow host ranges, the major cause of fermentation failure, due to their short latent period and large burst sizes. They are generally classified as Siphoviridae (having isometric heads and long, non-contractile tails) and usually fall into two groups (cos- and pac-type), based on their mode of DNA packaging and the number of major structural proteins present (Le Marrec et al., 1997. Applied and Environmental Microbiology 63 (8), p. 3246-3253—Two groups of bacteriophages infecting Streptococcus thermophilus can be distinguished on the basis of mode of packaging and genetic determinants for major structural proteins). More recently, a third group of phages infecting S. thermophilus was identified that represents a novel genetic lineage and highlights the genetic plasticity of these phages (Mills et al., 2011. International Dairy Journal 21, p. 963-969—A new phage on the ‘Mozzarella’ block: Bacteriophage 5093 shares a low level of homology with other Streptococcus thermophilus phages). Consequently, phages of S. thermophilus persist in dairy fermentation facilities leading to starter culture infections. In reponse to these infections, microorganisms such as S. thermophilus has evolved several mechanisms of phage resistance, some of which are more effective and stable than others. Mutants which have become resistant to phages by means of effective and stable mechanisms may be characterised by means of DNA sequencing, morphological analyses and/or adsorption assays.

Bacteriophage resistance systems have evolved in microorganisms such as S. thermophilus in tandem with phage adaptation strategies to overcome these biological barriers. These systems can include those preventing phage adsorption, blocking DNA injection, restriction/modification of DNA (R/M) and abortive infection or Abi (Labrie et al. (2010) Nature reviews 8, p. 317-327—Bacteriophage resistance mechanisms). To date, the most intensely characterised and the most frequent of these systems in lactic streptococci, are the Clustered Regularly Interspaced Short Palindromic Repeats (CRISPR) systems, which are known to provide acquired immunity to phages through an RNA-mediated dsDNA targeting process (Barrangou et al. (2007). Science 315, p. 1709-1712—CRISPR provides acquired resistance against viruses in prokaryotes; Garneau et al. (2010). Nature 468, p. 67-71—The CRISPR/Cas bacterial immune system cleaves bacteriophage and plasmid DNA).

Three distinct CRISPR systems (CRISPRs 1, 2 and 3), representing two distinct types (types II and III) are widespread in S. thermophilus and individual strains may contain multiple systems. Diversity was observed across three CRISPR loci between 124 different S. thermophilus strains. Specifically, CRISPR1 was ubiquitous, whereas CRISPR2 was present in 59 of 65 strains, and CRISPR3 was present in 53 of 66 strains. A total of 49 strains (39.5%) carried all three loci. (Horvath et al., 2008. Journal of Bacteriology 190 (4), p. 1401-1412—Diversity, activity, and evolution of CRISPR loci in Streptococcus thermophilus). Recently, a fourth CRISPR system has been described (Sinkunas et al., 2013. The European Molecular Biology Organisation journal 32, p. 385-394—In vitro reconstitution of cascade-mediated CRISPR immunity in Streptococcus thermophilus) although its prevalence is rare and in vivo activity is not known. Although CRISPR provides effective immunity against phages through acquired spacers which are identical to short regions of the attacking phage genomes (Barrangou et al., 2007, as above), it is known that phages can rapidly evolve to overcome these spacer additions through single nucleotide alterations in the corresponding genomic region (Deveau et al., 2008. Journal of Bacteriology 190 (4), p. 1390-1400—Phage response to CRISPR-encoded resistance in Streptococcus thermophilus). Furthermore, since CRISPR mutations are the most frequent mutations involved in phage resistance it is difficult to identify other more desirable mutations which provide phage resistance.

A more targeted approach is highly demanded to increase the occurrence of non-CRISPR BIMs by derivation protocols. WO2016/012552 describes selective inactivation of CRISPR immunity by silencing cas genes in the active CRISPR loci of S. thermophilus. However, with this approach the cas genes are silenced by introducing a plasmid expressing an antisense RNA directed towards the cas gene transcripts, therefore, making the lactic acid bacteria strain a genetically modified organism (GMO). WO2018/197495 describes a GMO-method to enrich for and obtain non-CRISPR BIMs by expressing anti-CRISPR protein genes in S. thermophilus. Currently, the use of GMO microorganisms is regulated strictly and prohibited by governments for food applications in the case of starter cultures.

It is desirable to develop a method to obtain phage-resistant derivatives of a lactic acid bacterium parent strain suitable for food and feed fermentation, and especially S. thermophilus, where such phage resistance is due to the action of alternative phage resistance mechanisms than CRISPR. The present invention provides a method to obtain such phage-resistant bacteria.

### FIGURES

FIG. 1—Representative double agar overlay spot assay on bacterial lawn of DS75685 (685; top panels) or DS75686 (686; bottom panels) transformants transformed with (from left to right): pNZ44, pNZ44-acr100E-D1 (acr100E-D1), pNZ44-acr200E-D77 (acr200E-D77), pNZ44-acr300F (acr300F) and pNZ44-acrIIA5 (acrIIA5). Phages spotted were from left to right: phage cocktail with four 100-E phages (PC), ϕ100E-D4A-L (D4), ϕ100E-D3A-L (D3), ϕ100E-D2A-L (D2) and ϕ100E-D1A-L (D1). Dilutions (10⁻¹ to 10⁻⁷) of phage stock were spotted from top to bottom on each agar plate (indicated on right of most right panel).

FIG. 2—Graph of double agar overlay spot assay on bacterial lawn of DS75685 (685) transformants transformed with (from left to right): pNZ44 (685::pNZ44), pNZ44-acr100E-D1 (685::acr100E-D1), pNZ44-acr200E-D77 (685::acr200E-D77), pNZ44-acr300F (685::acr300F) and pNZ44-acrIIA5 (685::acrIIA5). Each bar represents the average infection phage titer (pfu/mL) measured in triplicate. Error bars represent standard deviation. Values on y-axis are depicted on log scale.

FIG. 3—Representative double agar overlay spot assay on bacterial lawn of DS75685 (685) transformants cured from their respective plasmid (from left to right): pNZ44-acr100E-D1 (acr100E-D1), pNZ44-acr200E-D77 (acr200E-D77), pNZ44-acr300F (acr300F) and pNZ44-acrIIA5 (acrIIA5). Plasmid-cured transformants are boxed. Panel outside box represent spot assay on 685::acrIIA5 transformant (acrIIA5) with maintained plasmid. Phages spotted were from left to right: phage cocktail with four 100-E phages (PC), ϕ100E-D4A-L (D4), ϕ100E-D3A-L (D3), ϕ100E-D2A-L (D2) and ϕ100E-D1 A-L (D1). Dilutions (10⁻¹ to 10⁻⁸) of phage stock were spotted from top to bottom on each agar plate (indicated on right of most right panel).

### DETAILED DESCRIPTION

The inventors of the present invention surprisingly noted that using a method of the invention provides a higher chance to obtain a bacteriophage-insensitive mutant (BIM) whereby the phage resistance is conferred at least by a mechanism other than CRISPR and the acidification performance is maintained or improved when compared to the acidification performance of the parent strain without having to test a multitude of BIMs to secure both traits. In alternative wording: a method of the invention provides an enrichment of non-CRISPR or double hurdle BIMs with maintained acidification performance compared to the parent reference strain.

With current BIM derivation protocols there is no means of ensuring for enrichment for non-CRISPR BIMs with maintained dairy application performance traits such as texture, acidification rate, maintained post-acidification phenotype. Most apparent traits are acidification properties under varying conditions (e.g. different temperatures ranging for S. thermophilus strains from 35-42° C.). Important properties related to acidification are specific acidification rate, and, thereby, TTR (time to reach) a certain target pH relevant for the specific application (whether it would be for e.g. mozzarella, cottage cheese, continental or swiss cheese applications). Also, a stable post-acidification behaviour of a starter culture is valued by the industry, for instance, for yoghurt applications a stable post acidification phenotype during storage of product. Since the survival of mutants in BIM derivation strategies is dependent on the acquisition of spontaneous mutations (such as spacer addition in CRISPR loci or mutations in coding sequences resulting in non-CRISPR mediated phage resistance), the nature of the mutations could be beneficial for phage resistance but at the same time disadvantageous for application performance. Therefore, in current screens for suitable BIMs of S. thermophilus strains a substantial number of BIMs have to be screened to select for the one mutant that is a broad and stable phage-insensitive phenotype, and at the same time has a maintained application-relevant phenotype. Especially, this is the case in most corporate classical strain improvement programs in which chemical, radioactive isotope- or UV-mediated mutagenesis is applied to increase the diversity of mutations in the batch of S. thermophilus cells.

The methods of the present invention overcome these problems. A method of the invention provides an increased chance that BIMs with mutations beneficial for phage insensitivity and maintained acidification characteristics on milk become more prominent in the culture and are enriched for. In this way, the success rate increases to isolate BIMs with both desired features, i.e. phage insensitivity and maintained acidification properties in milk.

The approach of the claimed method makes use of a BIM derivation method enriching for both non-CRISPR BIMs and mutants with maintained acidification performance on non-GMO basis.

Thereby, market acceptance of resulting BIMs in commercial starter cultures is ensured. Since the method directs towards enrichment for desired mutants on all desired traits, one could apply mutagenesis to increase diversity of mutations without the need to screen for a substantial higher number of BIMs. Throughout the method an enrichment of non-CRISPR BIMs is obtained by using (a) virulent phage(s) expressing functional anti-CRISPR protein-encoding genes for exposing S. thermophilus strains. Subsequently, the entire population of BIMs is subjected to several passages in milk-based medium while exposing the cultivations either continuously or in alternating cycles to at least the anti-CRISPR protein-expressing phage. Optionally, additional virulent phages can be added to the anti-CRISPR protein-expressing phage during cultivation cycles under application-relevant conditions to enrich for broader resistance to a diversity of virulent phages if present against a specific host. These cultivation cycles under exposure of phage with anti-CRISPR activity enriches the population of BIMs for non-CRISPR mediated resistance and maintained application performance traits. Optionally, cultivation cycles can be modified towards the application BIMs are derived for. For example, alternation of cultivation cycles can be applied: with or without phage challenge (to allow for enrichment solely on fermentation characteristics), temperature, medium, carbon source.

The invention provides a method for obtaining a non-CRISPR bacteriophage-insensitive mutant (BIM) from a lactic acid bacterium parent strain comprising
a. exposing said parent strain to at least one virulent bacteriophage which expresses a gene encoding an anti-CRISPR protein
b. growing surviving lactic acid bacteria for at least one cycle in milk in the presence of said at least one bacteriophage which expresses a gene encoding an anti-CRISPR protein
c. isolating BIMs resulting from step b
d. characterizing at least one isolated BIM by
   i. exposing said isolated BIM to at least two different bacteriophages to determine the spectrum of bacteriophage insensitivity, and
   ii. testing the stability of the acquired bacteriophage resistance of said at least one isolated BIM, and
   iii. determining the acidification profile of said isolated BIM in milk
e. and selecting a BIM which
   i. when compared to the parent strain is insensitive to more bacteriophages as determined in step d (i), and
   ii. when compared to the parent strain has an improved stability of the acquired bacteriophage resistance as determined in step d(ii), and
   iii. has an acidification profile, as determined in step d(iii), in milk which is at least comparable to or improved when compared to said parent strain.

As used herein, the term “lactic acid bacteria or lactic acid bacterium” (LAB) refers to food-grade bacteria producing lactic acid as the major metabolic end-product of carbohydrate fermentation. These bacteria are related by their common metabolic and physiological characteristics and are usually Gram positive, low-GC, acid tolerant, non-sporulating, non-respiring, rod-shaped bacilli or cocci. During the fermentation stage, the consumption of lactose by these bacteria causes the formation of lactic acid, reducing the pH and leading to the formation of a protein coagulum. These bacteria are thus responsible for the acidification of milk and for the texture of the dairy product. As used herein, the term “lactic acid bacteria or lactic acid bacterium” encompasses, but is not limited to, bacteria belonging to the genus of Lactobacillus spp., Bifidobacterium spp., Streptococcus spp., such as Lactobacillus delbruekii subsp. bulgaricus, Streptococcus salivarius thermophilus, Bifidobacterium animalis, Lactobacillus casei, Lactobacillus plantarum, Lactobacillus helveticus, Lactobacillus acidophilus and Bifidobacterium breve.

The phrase “a non-CRISPR bacteriophage-insensitive mutant (BIM)” as used herein refers to a BIM wherein the phage resistance is conferred by a mechanism other than CRISPR.

The phrase “bacteriophage which expresses a gene encoding an anti-CRISPR protein” is used herein to refer to a bacteriophage which comprises genetic information encoding an anti-CRISPR protein. Preferably, the anti-CRIPR protein is expressed in said bacteriophage and hence said bacteriophage comprises an anti-CRISPR protein.

The term “anti-CRISPR protein” refers to a protein which interferes with a function of a bacterial CRISPR-Cas system involved in the immunity of a bacterial strain against virulent bacteriophages. Such proteins can interfere, (physically) interact or lower the activity of the different components of the CRISPR-Cas system. The components can play a role in the adaptation, or interference phase of the CRISPR-Cas system. Such anti-CRISPR proteins are encoded by so-called “anti-CRISPR protein encoding genes”. These genes can be found in the genomes of virulent lytic bacteriophages or prophages. In the case of prophages, the anti-CRISPR protein encoding gene can therefore be also found in the genome of a lactic acid bacterium.

Anti-CRISPR proteins interfere, interact or lower activity of CRISPR-Cas systems of class 1 or class 2 system. More specifically, anti-CRISPR proteins interfere, interact or lower activity of

CRISPR-Cas systems of class 1 or class 2 system of Lactobacilli spp. Even more, anti-CRISPR proteins interfere, interact or lower activity of CRISPR-Cas systems of class 2 of Streptococcus spp., even more specifically class 2 systems of Streptococcus thermophilus.

Bacteriophages bearing anti-CRISPR protein encoding genes in their genome can be found by sequencing the genomes of phages and mining genome sequences for known anti-CRISPR protein encoding gene sequences. Anti-CRISPR proteins AcrIIA5 and AcrIIA6 were described in S. thermophilus phages (Hynes et al., 2018, Nature Communications, 9, 2919). A person skilled in the art can find anti-CRISPR protein expressing phages by sequencing for example S. thermophilus phage genomes by e.g. Illumina or Nanopore (Oxford) sequencing methods, and mining the genome sequences with BLAST searches to look for orthologs of known anti-CRISPR proteins, such as AcrIIA5 and AcrIIA6 (Hynes et al., 2018, Nature Communications, 9, 2919) or other anti-CRISPR protein gene sequences in the phage genomes. With a similar approach, in a screen of 254 S. thermophilus phage genomes, Hynes et al. (2018, Nature Communications, 9, 2919) found that 35% contained an acrIIA6 ortholog and 5.5% contained an acrIIA5 ortholog with at least 75% sequence identity over 75% of the gene length. Furthermore, WO2018/197495 describes 233 potential anti-CRISPR protein encoding genes grouped in 84 families by in silico analysis using conserved neighbourhood analysis followed by restriction on predicted protein size and elimination of genes with known function.

One can screen for bacteriophages bearing anti-CRISPR protein encoding genes similar as described by Hynes et al. 2017, by providing a lactic acid bacterium strain immunized against virulent phages by plasmid programming resulting in the addition of (a) conservative spacer(s) against all said virulent bacteriophages in the CRISPR loci of said lactic acid bacterium strain, and screening said bacteriophages against the CRISPR-immunized lactic acid bacterium by determining infection titers (according to standard techniques). The bacteriophage which can build up infection titers against CRISPR-immunized lactic acid bacterium strain comparable to the sensitive lactic acid bacterium strain are bacteriophages which could encompass an anti-CRISPR protein encoding gene. Coding gene sequences displaying homology with known anti-CRISPR protein encoding genes from the selected bacteriophage can be screened by expressing said genes on plasmid in CRISPR-immunized lactic acid bacterium strain, exposing to any other virulent bacteriophage and determining whether expression of gene sequences from selected bacteriophage result in regained sensitivity of CRISPR-immunized lactic acid bacterium to other virulent bacteriophage. If regained sensitivity is observed, this indicates anti-CRISPR activity of said gene, and indicates that selected bacteriophage can be utilized as anti-CRISPR protein-bearing bacteriophage for BIM derivation protocols.

Step (a)—Exposing the parent strain to at least one virulent (for said parent strain) bacteriophage, may be carried out in any suitable medium, for instance in an aqueous solution such as a buffered aqueous solution or in a soft agar medium or in milk. In a preferred embodiment, exposing the parent strain to a bacteriophage is carried out in a soft agar medium. In another preferred embodiment, exposing the parent strain to a bacteriophage is carried out in milk. The milk may be incubated overnight or until clotting is observed. The parent strain used in the method of the invention may be pre-treated in order to increase the genetic diversity and to increase the number of the BIMs. This pre-treatment may be carried out by methods known in the art, such as chemical mutagenesis or by irradiation with UV-light. I.e. preferably, the lactic acid bacterium parent strain used in step (a) is mutagenized. The—optionally pre-treated—parent strain may be exposed to one type of bacteriophage or to multiple different bacteriophages, for instance to 2, 3, 4 or 5 different bacteriophages.

When step (a) is performed on a soft agar medium, surviving colonies of the lactic acid bacterium are optionally pooled before performing step (b). The pooled surviving colonies may optionally be grown in a suitable (liquid) medium before performing step (b).

When step (a) is performed in an aqueous solution, surviving lactic acid bacteria may optionally be grown in a suitable (liquid) medium before performing step (b).

Preferably, step (a) comprises exposing said parent strain to at least two different virulent bacteriophages and wherein at least one of said two different bacteriophages is a virulent bacteriophage which expresses a gene encoding an anti-CRISPR protein.

Step (b)—growing surviving lactic acid bacteria for at least one cycle in milk in the presence of said at least one bacteriophage which expresses a gene encoding an anti-CRISPR protein, may be carried out on surviving colonies or on surviving lactic acid bacteria obtained a step (a). Alternatively, step (b) is performed on surviving colonies or surviving lactic acid bacteria which have been allowed to growth on suitable media as explained in the preceding 2 paragraphs describing said optional growth.

Step (b) allows for enrichment of BIMS which have a performance in milk which is desired in an industrial setting. Essentially only the lactic acid bacteria which properly grow in milk in the presence of at least one bacteriophage which expresses a gene encoding an anti-CRISPR protein, will grow.

Preferably, step (b) comprises at least 2, 3, 4, 5, 6, 7, 8, 9 or 10, preferably at least 15, more preferably at least 20, most preferably at least 25 cycles.

In case more than one cycle is used:
the first cycle comprises growing surviving lactic acid bacteria in milk in the presence of said at least one bacteriophage which expresses a gene encoding an anti-CRISPR protein
taking a sample which comprises lactic acid bacteria and bacteriophages from said incubated milk
growing the obtained sample in a new batch of milk and repeating the taking of a sample and growing of the last obtained sample in a new batch of milk
optionally, adding a new batch of bacteriophages to a new batch of milk The lactic acid bacteria and the bacteriophage are co-evolving in the above described steps.

Step (c)—isolating BIMs resulting from step b is carried out such as to isolate single colonies of one or more bacteriophage insensitive mutant. The incubated (clotted) milk obtained in step (b) may be plated on agar plates. After incubating the agar plates at a temperature at which the lactic acid bacterium (preferably S. thermophilus) may grow, colonies may appear which represent the BIMs. The colonies may be purified and preferably phenotypically verified to obtain a single strain BIM according to methods known in the art.

Step (d)—characterization of at least one BIM is performed by performing at least 3 different analysis:
i. exposing said isolated BIM to at least two different bacteriophages to determine the spectrum of bacteriophage insensitivity, and
ii. testing the stability of the acquired bacteriophage resistance of said at least one isolated BIM, and
iii. determining the acidification profile of said isolated BIM in milk Step (d) (i) is performed such as to determine the broadness of the acquired resistance and is typically performed by exposing the isolated BIM to at least two different bacteriophages.

This may be carried out in any suitable medium, for instance in an aqueous solution such as a buffered aqueous solution or in a soft agar medium or in milk. In a preferred embodiment, exposing the BIM to at least two different bacteriophages is carried out in a soft agar medium. In another preferred embodiment, exposing the BIM to at least two different bacteriophages is carried out in milk. The milk may be incubated overnight or until clotting is observed. Preferably, the lactic acid bacterium parent strain is taken along as a comparison. The used bacteriophages are typically bacteriophages which are virulent for the lactic acid bacterium parent strain.

Upon comparing the BIM and the lactic acid bacterium parent strain, the BIM is typically resistant to the tested bacteriophages whereas the lactic acid bacterium parent is sensitive to the same bacteriophage.

Step (d)(ii) is performed such as to determine ability of the BIM to survive multiple cycli of (evolving) bacteriophages. More detailed information in respect of this step is given below as well as in the experimental part herein.

Step (d)(iii) is performed such as to determine whether the BIM has an acidification profile in milk similar to the lactic acid bacterium parent strain. Suitable parameters which can be taken into account are the time to reach a certain pH, such as pH 4.6. Preferably the time to reach the desired pH (for example 4.6) in milk is identical or similar or faster when compared to the time to reach the same pH of the lactic acid bacterium parent strain.

The term time to reach a desired pH such as pH 4.6 is defined as the time between start of fermentation of a milk substrate with lactic acid bacteria, or contacting a milk substrate with lactic acid bacteria, until a milk substrate with a desired pH such as a pH of 4.6 is reached.

Step (e)—selecting at least one BIM which:
i. when compared to the parent strain is insensitive to more bacteriophages as determined in step d (i), and
ii. when compared to the parent strain has an improved stability of the acquired bacteriophage resistance as determined in step d(ii), and
iii. has an acidification profile, as determined in step d(iii), in milk which is at least comparable to or improved when compared to said parent strain.

Although not required, one may want to confirm that the obtained BIM is indeed a non-CRISPR BIM by analysing the CRISPR loci. Preferably, in the present context, the term CRISPR loci means the loci of the CRISPR system 1, 2 and 3. A non-CRISPR BIM will comprise CRISPR loci which are identical to the CRISPR loci of the lactic acid bacterium parent strain. The advantage of a method of the invention is that the selected BIMs have become phage resistant by means of a mechanism that is different from CRISPR and therefore based on an alternative phage resistance mechanism. As a result, the BIMs obtained by the method of the invention may have a more stable and/or robust phage resistance compared to a CRISPR-mediated BIM of which it is known that phages can rapidly evolve to overcome these spacer additions through single nucleotide alterations in the appropriate genomic region. In case one would like to verify that the BIMs have acquired their phage resistance due to a mechanism different from the CRISPR resistance mechanism, additional steps can be performed such as:
comparing the CRISPR loci of the parent strain with the CRISPR loci of the bacteriophage insensitive mutant and
select the bacteriophage insensitive mutant of which the CRISPR loci is identical to the CRISPR loci of the parent strain.

Step (d) (ii) can be performed in many ways, for example by comprising the steps of:
A. growing an isolated BIM in milk in the presence of a bacteriophage
B. obtaining bacteriophages from the resulting milk of step A, and
C. growing said isolated BIM in milk in the presence of newly added bacteriophage and the bacteriophages obtained in step B,
D. repeating obtaining bacteriophages from the resulting milk and growing of said isolated BIM in milk in the presence of newly added bacteriophage and the obtained bacteriophages for at least a total of 3 cycles,
to determine the number of cycles of survival of an isolated BIM as a measure for stability of insensitivity in batch cultures with evolving bacteriophage present

Essentially, the isolated BIM is challenged with an evolving bacteriophage to which resistance was acquired in step (a) or steps (a) and (b).

The use of a total of 3 cycles is sufficient to discriminate between CRISPR and non-CRISPR BIMs. Additional cycles can be used to obtain BIMs with improved bacteriophage survival.

Preferably, the amount of cycles in step d(ii)(D) comprises at least 5 or 10, preferably at least 15, more preferably at least 20, most preferably at least 25 cycles. A BIM is more robust if it can survival a higher number of cycles.

As outlined, above, the term “lactic acid bacteria or lactic acid bacterium” encompasses, but is not limited to, bacteria belonging to the genus of Lactobacillus spp., Bifidobacterium spp., Streptococcus spp., Lactococcus spp., such as Lactobacillus delbruekii subsp. bulgaricus, Streptococcus salivarius thermophilus, Bifidobacterium animalis, Lactobacillus casei, Lactobacillus plantarum, Lactobacillus helveticus, Lactobacillus acidophilus and Bifidobacterium breve. Preferably, a method of the invention is performed on a Streptococcus thermophilus strain. I.e. the invention preferably provides a method for obtaining a non-CRISPR bacteriophage-insensitive mutant (BIM) from a Streptococcus thermophilus parent strain comprising
a. exposing said parent strain to at least one virulent bacteriophage which expresses a gene encoding an anti-CRISPR protein
b. growing surviving Streptococcus thermophilus bacteria for at least one cycle in milk in the presence of said at least one bacteriophage which expresses a gene encoding an anti-CRISPR protein
c. isolating BIMs resulting from step b
d. characterizing at least one isolated BIM by
   i. exposing said isolated BIM to at least two different bacteriophages to determine the spectrum of bacteriophage insensitivity, and
   ii. testing the stability of the acquired bacteriophage resistance of said at least one isolated BIM, and
   iii. determining the acidification profile of said isolated BIM in milk
e. and selecting a BIM which
   i. when compared to the parent strain is insensitive to more bacteriophages as determined in step d (i), and
   ii. when compared to the parent strain has an improved stability of the acquired bacteriophage resistance as determined in step d(ii), and
   iii. has an acidification profile, as determined in step d(iii), in milk which is at least comparable to or improved when compared to said parent strain.

The lactic acid bacterium or preferably the Streptococcus thermophilus parent strain used in any of the above described methods of the invention, may either be sensitive or be insensitive to the bacteriophage to which the parent strain is exposed, such as insensitive due to the previous addition of a CRISPR spacer. In the case of a sensitive parent strain, inactivation of the CRISPR defence mechanism by the anti-CRISPR protein prevents the formation of CRISPR BIMs in the step involving the exposure to at least one bacteriophage. In the case of the insensitive parent strain (such as due to the previous addition of a CRISPR spacer), the parent strain is first made sensitive by the anti-CRISPR protein which then also prevents the formation of phages that have overcome the CRISPR-mediated defence mechanism in the step involving the exposure to at least one bacteriophage. This may lead to a BIM which is resistant to one or more phages by both CRISPR and non-CRISPR mechanisms.

Further optional steps to any of the above described methods are:
further comprising adding a cryoprotectant to the obtained bacteriophage insensitive mutant, or
further comprising freeze drying or freezing the obtained bacteriophage insensitive mutant.

BIMs display phage insensitivity by CRISPR immunity (CRISPR BIMs) through additions of spacers against previously infecting phages, or non-CRISPR immunity (non-CRISPR BIMs) by other mutations, e.g. in receptors of other docking sites for bacteriophages. CRISPR immunity tends to be overcome by evolving phages in a dairy plant, e.g. by acquiring point mutations in sequences part of the CRISPR spacers in the host. Non-CRISPR immunity (possibly additionally to CRISPR immunity), however, results in prevention of docking of phages on the cell wall, or prevention of DNA injection, which is a more difficult hurdle for evolving phage to overcome. Therefore, non-CRISPR BIMs are considered more phage robust, i.e. a broader range of insensitivity and longer period of insensitivity against evolving phage population in dairy plant. A BIM with both acquired CRISPR and non-CRISPR immunity could be as effective and is named a ‘double hurdle BIM’.

Preferably, the present method, including the disclosed embodiments, further comprises culturing the one or more selected bacteriophage insensitive mutant in a culture medium, and/or recovering the bacteriophage insensitive mutant from the culture medium to provide a starter culture composition. “Starter culture” is defined herein as a preparation containing microbial cells that is intended for, or suitable for, inoculating a medium to be fermented. Such Starter cultures are generally referred to as direct vat set (DVS) or direct-to-vat inoculation (DVI) cultures or bulk starter cultures. The provision of a starter culture is advantageous since starter cultures can be inoculated directly into milk without intermediate transfer and/or propagation. Preferably, culturing is carried out at conditions such as temperature and pH control conducive to the growth of the microorganisms, or preferably S. thermophilus for a period of time until the desired cell concentration and activity of the culture are reached. The skilled person is able to determine the correct conditions for culturing S. thermophilus, or the desired microorganism.

Preferably, to the present bacteriophage insensitive mutant, or to the starter culture composition, an additive is added. For example, a cryoprotectant is added. A “cryoprotectant” is defined herein as a substance used to protect cells or tissues from damage during freezing and thawing. The cryoprotectant may be any additive as long as it protects cells or tissues from damage during freezing and thawing.

Examples of cryoprotectants include, but are not limited to, sugars (e.g. sucrose, fructose, trehalose), polyalcohols (e.g. glycerol, sorbitol, mannitol), polysaccharides (e.g. celluloses, starch, gums, maltodextrin), polyethers (e.g. polypropylene glycol, polyethylene glycol, polybutylene glycol), antioxidants (e.g. natural antioxidants such as ascorbic acid, beta-carotene, vitamin E, glutathione, chemical antioxidants), oils (e.g. rapeseed oil, sunflower oil, olive oil), surfactants (e.g. Tween®20, Tween®80, fatty acids), peptones (e.g. soy peptones, wheat peptone, whey peptone), tryptones, vitamins, minerals (e.g. iron, manganese, zinc), hydrolysates (e.g. protein hydrolysates such as whey powder, malt extract, soy), amino acids, peptides, proteins, nucleic acids, nucleotides, nucleobases (e.g. cytosine, guanine, adenine, thymine, uracil, xanthine, hypoxanthine, inosine), yeast extracts (e.g. yeast extracts of Saccharomyces spp., or Torula spp.), beef extract, growth factors, and lipids. Preferably, the present method further comprises a step of freeze drying or freezing the present bacteriophage insensitive mutant. More preferably freeze drying to provide a dry powder. Alternatively freezing to provide a frozen matrix, such as frozen pellets. Freeze-drying is a technique well known in the art and may comprise the steps of freezing microorganisms to get frozen material and subsequently reducing the surrounding pressure while adding enough heat to allow the frozen water in the frozen material to sublime directly from the solid phase into the gas phase. Freeze-drying equipment that can be used includes, but is not limited to, rotary evaporator freeze-driers, manifold freeze-driers and tray freeze-driers. If necessary, a secondary step can be performed that aims to remove unfrozen water molecules. It is well within the experience of the person skilled in the art to establish a suitable temperature and pressure profile to achieve satisfactory freeze-drying. The freeze-dried material can be a powder or a granule.

In yet another aspect, the invention relates to a starter culture composition comprising the herein described bacteriophage insensitive mutant. Preferably, the starter culture composition is suitable for inoculation of a medium to be fermented on an industrial scale. Preferably the starter culture composition is suitable for inoculation of milk for the production of fermented milk products. More preferably the starter culture composition comprises an additive. An example of an additive is a cryoprotectant. Additionally, the starter culture composition may comprise other microorganisms or other lactic acid bacteria such as lactic acid bacteria belonging to the genera Lactococcus, Leuconostoc or Lactobacillus. More preferably the starter culture composition comprises a combination of the present bacteriophage insensitive mutant with L. bulgaricus, or Lactobacillus delbrueckii subsp. bulgaricus. Such a mixed starter culture is advantageous for the provision of yoghurt. Alternatively, for the provision of cheese, the present starter culture composition comprises a combination of the present bacteriophage insensitive mutant with Lactococcus lactis.

Preferably, the starter culture composition is frozen, preferably in the form of frozen pellets such as individual frozen pellets. Preferably the frozen pellets comprise as additive formate, such as sodium formate. Preferably the present frozen pellets have an average diameter within the range of 0.1 to 10 mm. The advantage of frozen pellets is that they will not stick and flow freely which allows a convenient dosing of the frozen pellets. Preferably the frozen pellets comprise a content of viable bacteria, preferably S. thermophilus, of at least 10⁹ colony forming units (CFU) per gram frozen pellets. The advantage of such concentrated frozen material is that only low amounts of frozen material is necessary to inoculate milk in industrial milk fermentation processes.

Alternatively, the starter culture composition is freeze-dried. A freeze-dried starter composition may be in the form of a pellet, granule, tablet or a powder. Most preferably as a powder. The freeze-dried culture compositions can be stored and transported without refrigeration for extended periods of time under dry conditions. However, storage below 0° C. is recommended, more preferably below 15° C.

Alternatively, the present starter culture composition may be in liquid form.

In yet another aspect, the invention relates to a container comprising the present bacteriophage insensitive mutant or comprising the present starter culture composition. The advantage of packing the present bacteriophage insensitive mutant or starter culture composition in a container is the ease of storage and transport. Preferably the present container is a commercial relevant package. An example of a commercial relevant package is a container comprising at least 50 or 500 gram frozen material when formulated in a frozen form, or comprising at least 50, 200 or at least 500 gram when formulated in a freeze-dried form.

The invention thus also provides a starter culture composition suitable for inoculation of a medium to be fermented on an industrial scale comprising a bacteriophage insensitive mutant as obtained by any of the herein described methods. In a preferred embodiment, the starter culture composition is frozen, freeze dried or in liquid form.

In yet another aspect, the invention provides a process for the production of a dairy product such as a fermented milk product or cheese comprising the use of one or more of the bacteriophage-insensitive mutant of the lactic acid bacterium, preferably Streptococcus thermophilus, parent strain as disclosed hereinbefore or comprising the use of a starter culture composition as disclosed hereinbefore.

In a further aspect, the invention provides the use of the bacteriophage-insensitive mutant of the lactic acid bacterium, or preferably of Streptococcus thermophilus, parent strain as disclosed hereinbefore or use of a starter culture composition as disclosed hereinbefore in a process for the production of a dairy product, such as a fermented milk or cheese.

The invention will be explained in more detail in the following example, which are not limiting the invention.

### Material and Methods

### 1. Bacterial Growth Conditions

Individual strains of S. thermophilus (as indicated in Table 1) are routinely grown from 10% Reconstituted Skimmed Milk (RSM) stocks, 20% glycerol stocks (Sigma Aldrich, Germany) or from a single colony overnight (ON) at 42° C. in Phage Detection Medium (PDM: 10g/L yeast extract, 6 g/L bactopeptone, 10 g/L Na-β-glycerophosphate, 1 mM MgSO₄) supplemented with 0.5% lactose, or on plates containing 10 g/L bacto agar (PDM-agar).

For bacteriophage assays: firstly, a solid bottom agar is prepared with PDM agar supplemented with 0.5% glycine either in petridishes (20 mL) or omnitrays (45 mL; NUNC, VWR);

secondly, an overnight (ON) culture is prepared by inoculating 2% from bacterial stock in 10% RSM and incubating ON at 42° C.; thirdly, a top agar is prepared by mixing prewarmed PDM broth with molten PDM agar supplemented with 0.5% glycine at a ratio of 2:1, subsequently using 3.6 mL of semi-solid top agar with 0.4 mL ON culture of the appropriate S. thermophilus host to pour onto the bottom agar resulting in double agar plates. All transformants are maintained as above with the addition of chloramphenicol (Sigma-Aldrich, Germany) to a final concentration of 5 micrograms/mL (PDM-Cm5).

For E. coli cultivation, 2*PY medium (16 g/L phytone peptone, 10 g/L yeast extract (Difco), 5 g/L NaCI) is used. For solid medium (2*PY-agar), 15 g/L granulated agar is added.

### 2. Propagation of Bacteriophages

The previously isolated virulent bacteriophages against S. thermophilus 100-E (see Table 1) are described in WO2016/012552. Bacteriophages are propagated as follows: 20 ml PDM broth is inoculated (2%) with a fresh ON culture of the appropriate host strain and incubated at 42° C. for one or two hours at which point CaCl₂ is added to a final concentration of 10 mM, and 400 microliters of the bacteriophage stock solution. The suspension is mixed well and incubated for a further 2-4 hours until visible lysis of the culture is observed. The lysed culture is centrifuged and the supernatant filtered (0.45 micrometers pore size). The filtered supernatant is used as the phage stock for subsequent assays.

TABLE 1
List of plasmids, bacterial strains and phages used

| Plasmid | Description | Reference |
|---|---|---|
| pNZ44 | Transformation vector | McGrath et al., 2001, Appl |
| | | Environ Microbiol, 62, 608-616, |
| | | SEQ ID NO: 1 |
| pNZ44-acr100E-D1 | pNZ44-derived vector expressing acr100E- | Example 1 |
| | D1 | |
| pNZ44-acr200E-D77 | pNZ44-derived vector expressing acr200E- | Example 1 |
| | D77-D1 | |
| pNZ44-acr300F | pNZ44-derived vector expressing acr300F | Example 1 |
| pNZ44-acrIIA5 | pNZ44-derived vector expressing acrIIA5 | Example 1 |
| pNZ44-acrIIA6 | pNZ44-derived vector expressing acrIIA6 | Example 1 |
| Strain | Description | Reference |
| 100-E^{a} | Streptococcus thermophilus parent strain | WO2016/012552 |
| DS75685 | CRISPR BIM derived from 100-E | Example 1 |
| DS75686 | Double hurdle BIM derived from 100-E | Example 1 |
| 685::pNZ44 | Empty plasmid control transformant | Example 1 |
| | DS75685 | |
| 686::pNZ44 | Empty plasmid control transformant | Example 1 |
| | DS75686 | |
| 685::acr100E-D1 | pNZ44-acr100E-D1 transformant DS75685 | Example 1 |
| 686::acr100E-D1 | pNZ44-acr100E-D1 transformant DS75686 | Example 1 |
| 685::acr200E-D77 | pNZ44-acr200E-D77 transformant DS75685 | Example 1 |
| 686::acr200E-D77 | pNZ44-acr200E-D77 transformant DS75686 | Example 1 |
| 685::acr300F | pNZ44-acr300F transformant DS75685 | Example 1 |
| 686::acr300F | pNZ44-acr300F transformant DS75686 | Example 1 |
| 685::acrIIA5 | pNZ44-acrIIA5 transformant DS75685 | Example 1 |
| 686::acrIIA5 | pNZ44-acrIIA5 transformant DS75686 | Example 1 |
| DS68802^{b} | Streptococcus thermophilus parent strain | Example 2 |
| NEB10-bèta | High efficiency NEB ® 10-beta competent E. | New England Biolabs |
| | coli | |
| Phage | Description | Origin/reference |
| ϕ100E-D1A-L | Virulent phage of S. thermophilus 100-E | WO2016/012552, Example 1 |
| ϕ100E-D2A-L | Virulent phage of S. thermophilus 100-E | WO2016/012552, Example 1 |
| ϕ100E-D3A-L | Virulent phage of S. thermophilus 100-E | WO2016/012552, Example 1 |
| ϕ100E-D4A-L | Virulent phage of S. thermophilus 100-E | WO2016/012552, Example 1 |
| ϕ643-01 | Virulent phage of S. thermophilus DS68802 | Dairy plant, USA, Example 2 |
| ϕ643-03 | Virulent phage of S. thermophilus D568802 | Dairy plant, USA, Example 2 |
| ϕ643-11 | Virulent phage of S. thermophilus D568802 | Dairy plant, USA, Example 2 |
| | encoding AcrIIA5 ortholog AcrIIA5643 | |

^{a}Streptococcus thermophilus 100-E = D564900 was deposited on 15 July 2014 with the Centraal Bureau for Schimmelcultures, Uppsalalaan 8, 3508 AD in Utrecht, The Netherlands, and received deposition number CB5138555.
^{b}Streptococcus thermophilus strain D568802 is also known as commercial DSM product DELVO ® TEC TS-643

3. Derivation of BIMs from S. thermophilus

Firstly, S. thermophilus cells are optionally mutagenized as follows: 150 mL of ON culture is concentrated to 30 mL to increase cell density for sonication. Subsequently, cells are sonificated to break up chains according to standard procedures for S. thermophilus strains. After centrifugation and resuspending cells in 0.5 M Tris-maleate buffer, cells are exposed to either 0.005, 0.010 or 0.025 micrograms/mL of N-methyl-N-nitro-nitroso-guanidine (NTG, Sigma-Aldrich), along with one negative control that is not exposed to the NTG. Upon inactivation of the mutagen, with 10% w/v sodium thiosulphate, and washing twice with the 50 mM phosphate buffer, the cell pellets from each condition are resuspended in 5 mL phosphate buffer. From each mutagenized cell batch, 900 microliters are added to 1 mL of 20% glycerol which is then stored at −80° C. After routinely determining survival rates by plating of mutagenized cell batches, 250 microliters of selected batches (diluted to 10⁶ cfu/mL) are used in 10 mL of top agar medium supplemented with 100 microliters of 1M CaCl₂ and 500 microliters of bacteriophage stock (with a titre of approximately 10⁷ pfu/mL keeping to a cell:phage ratio of 1:10). The top agar overlay is incubated at 42° C. until visible surviving colonies can be counted and separated. Colonies, representing potential BIMs, growing in the top layer are twice single colony-purified, grown in 10% RSM, glycerol added, and stored at −80° C. for further characterization studies (as described below), or the whole soft agar overlay containing the potential BIMs is removed with a sterile spatula and incubated overnight in 10% RSM at 42° C.

The resulting clotted milk sample from is then continuously inoculated for at least one, more preferably five, even more preferably ten, most preferably more than twenty passages in 5 mL of 10% RSM at a 1% inoculum (10⁷ cfu/ml) in the presence of the anti-CRISPR protein expressing phage with or without more virulent phages to the passaged to the original S. thermophilus strain at an M.O.I. of 10 (10⁸ pfu/ml) (Mills et al., 2007, Journal of Microbiological Methods, 70, pp. 159-164). After the final passage, the clotted milk is inoculated onto solid PDM agar, and individual colonies are twice single colony-purified, grown in 10% RSM, glycerol added, and stored at −80° C. for further characterization studies (as described below).

### 4. Heap Lawrence Assay

The stability of the phage insensitivity is assessed by using a so called “Heap-Lawrence” assay as follows: stock solutions of the strains to be assessed (wild type plus BIMs) are made by inoculating 30 microliters of an overnight culture of the BIMs into 270 microliters of 10% RSM, growing overnight at 42° C. and then resuspending the culture with 1200 microliters of 10% RSM supplemented with 0.6% bromocresol purple (BCP; Sigma-Aldrich). The 200 microliters of the resuspended culture is aliquoted into new 96-well plates and stored at −20° C. for the Heap Lawrence assay. One of the plates from the −20° C. stock is incubated at 42° C. overnight. In 96 well deep well plates containing 600 microliters of 10% RSM+0.6% BCP, 75 microliters of the overnight culture is inoculated, along with 75 microliters of 5 times diluted bacteriophage solution if either a single phage isolate or a mixture of phages (a positive control is included where only the overnight BIM culture is inoculated without the presence of the bacteriophage). Following overnight incubation, the plates are evaluated for clotting and coloration due to the pH-dependent color of BCP. Green color indicates that the BIM has failed to clot and acidify the media and, thus, is susceptible to the bacteriophage in the culture whereas yellow color indicates that the BIM has survived the bacteriophage predation and has successfully acidify the culture.

After each acidification cycle, well plates are centrifuged at 4,000 g for 10 minutes and 200 microliters of supernatant from each well is collected and pooled. A few drops of 1M lactic acid solution are added in order to induce clotting of milk, followed by re-centrifugation at 4,000×g for 5 minutes and filter sterilization (0.45 μm pore size) of the suspension. Finally, the pH is adjusted to 7, using 0.8M KOH. This suspension contains the now evolved bacteriophages against the isolated BIMs and is then used in combination with the original bacteriophage suspension for the second cycle of Heap Lawrence. These rounds of Heap Lawrence are continued until no BIMs are able to acidify the media. The higher number of Heap Lawrence cycles a BIM survives, the higher the stability of its phage sensitivity is presumed to be.

### 5. Bacteriophage Assays

In order to enumerate the titer produced through phage propagation a spot overlay test is performed according to the following protocol. Firstly, the bacterial top agar containing the S. thermophilus strain to be assessed is prepared on PDM agar plates (as described above). From the propagated bacteriophage solution, serial dilutions are made (from 10⁻¹ to 10⁻⁸) and 10 microliters are spotted onto the top agar layer containing the S. thermophilus strains. After overnight incubation at 42° C., the titer of the propagated bacteriophage can be determined and expressed in plaque forming units per milliliter (pfu/mL) according to routine methods.

Subsequent spot assays to assay range of phage sensitivity of BIMs and transformants are performed on the abovementioned double agar medium in omnitrays or petridishes and applying 10 microliters of dilutions (10⁻¹ to 10⁻⁸) of a phage stock in a grid format, as described by Dupont, K., et al, J. (2005) (Detection of lactococcal 936-species bacteriophages in whey by magnetic capture hybridization PCR targeting a variable region of receptor binding protein genes. J Appl Microbiol, 98, 1001-1009). Plates are then allowed to dry and incubated ON at 42° C. The phage infection titre is determined from the highest dilution where a number of single plaques are observed. If no single plaques are observed the highest dilution with cell lysis is taken for calculation of phage titre. Efficiency of plaquing (EOP) is calculated by dividing the obtained titre of a given phage on the test strain by the titre of the same phage on the parent strain.

### 6. PCR Screening & CRISPR Locus Sequencing

All selected BIMs are subjected to PCR profiling to confirm their relatedness to the relevant parent strain from which they were derived, either by RAPD PCR or by CRISPR locus sequencing. RAPD PCR was performed on single colonies of each parent strain and BIM as template for the reaction and using the (GTG) 5′ RAPD profiling primer (Gevers, D., et aL, 2001. Applicability of rep-PCR fingerprinting for identification of Lactobacillus species. FEMS Microbiol Lett, 205, 31-36). The PCR conditions are as follows: 95° C.×10 min, followed by 30 cycles of 95° C.×15 s, 40° C.×30 s and 72° C. for 8 min with a final extension step of 72° C. for 16 min.

BIMs generated were purified and the CRISPR loci amplified by PCR and sequenced to determine acquisitions or alterations to the spacer content of the BIMs. CRISPR-1, CRISPR-2 and CRISPR-3 repeat/spacer arrays for each strain are amplified individually using a single colony of the appropriate strain as template material for the PCR and primers described previously by Horvath, P et aL (2008. Diversity, activity and evolution of CRISPR loci in Streptococcus thermophilus. Journal of bacteriology, 190, 1401-1412). The PCR conditions are as follows: 95° C.×10 min, followed by 30 cycles of 95° C.×15 s, 55° C.×15 s and 72° C. for either 2 min 45 s (CRISPR-1) or 1 min 10 s (CRISPR-2, and CRISPR-3) with a final extension step of 72° C. for 10 min. The PCR generated products are visualised on a 1% agarose (Fisher Scientific, USA) gel and purified using a PCR purification spin kit (Genomed, Germany).

Sanger sequencing (of all PCR products and plasmids) is performed in-house to verify the integrity of all plasmid constructs and to compare the sequences of the CRISPR loci of the BIMs to those of the corresponding parent strain. For CRISPR loci, this is performed by primer walking using synthetic primers based on a unique spacer of each repeat/spacer array in the internal regions of the sequences of the CRISPR loci, where required. CRISPRs are assembled using the Seqman program (DNAstar) and CRISPR arrays are visualised using the online CRISPR finder program (http://crispr.u-psud.fr).

7. Construction of Anti-CRISPR Protein Expressing Plasmid Vectors DNA sequences encoding three putative anti-CRISPR proteins acr100E-D1 (SEQ ID NO: 2), acr200E-D77 (SEQ ID NO: 3), acr300F (SEQ ID NO: 4), and two confirmed anti CRISPR proteins, acrIIA5 (SEQ ID NO: 5; Hynes et aL, 2017, Nat Microbiol, 2, 1374-1380), and acrIIA6 (SEQ ID NO: 6); Hynes et al., 2018, Nature Communications, 9, 2919) were prepared synthetically by DNA synthesis supplier as gBLOCKS (IDT, Belgium). Overhangs to pNZ44 were included for Gibson Assembly.

Cloning of anti-CRISPR protein DNA inserts into pNZ44 (SEQ ID NO: 1) is conducted with Gibson assembly. Gibson assembly enabled ligation of the pNZ44 plasmid to the insert by the use of gBlocks with added overhangs homologous to the Xbal restriction site on pNZ44. The Gibson reaction is performed using the Gibson Assembly HiFi 1-Step kit (Synthetic Genomics). Briefly, 0.04 pmols of insert along with 20ng of Xbal-digested pNZ44 is diluted in up to five microliters of milliQ. The GA HiFi 1 Step Master Mix (2×) is thawed on ice and vortexed before use. Five microliters of the master mix is added to each sample and mixed by pipetting. Samples are then incubated at 50° C. for one hour and then stored at −20° C.

pNZ44 and Gibson assembly reactions are transformed and amplified into NEB 10-beta competent E. coli (New England Biolabs, Table 1) according to the manufactures protocol. Transformants are plated onto 2*PY-agar plates supplemented with five micrograms/mL chloramphenicol and incubated ON at 37° C. Individual colonies are then picked and grown in five mL of 2*PY medium with five micrograms/mL chloramphenicol. The empty pNZ44 plasmid and clones of the Gibson assembly reactions are isolated and purified using Gene Jet plasmid Miniprep kit (Thermo Scientific, U.S.A.). Identity of the plasmid is then confirmed by restriction analysis, and correct clones are pooled and used for transformation of competent S. thermophilus cells.

8. Preparation of Competent S. thermophilus Cells

Competent cells are prepared as described by Holo & Nes (1989. High-Frequency Transformation, by Electroporation, of Lactococcus lactis subsp. cremoris Grown with Glycine in Osmotically Stabilized Media. Appl Environ Microbiol, 55, 3119-3123), with the following modifications: A series of tubes containing 50 mL PDM broth supplemented with 1% glycine (Sigma-Aldrich, Germany) and 0.3 M sucrose are prepared and inoculated (1%) with a fresh ON culture. The tubes are incubated at 42° C. until an OD600 of 0.5 was reached. Ampicillin (Sigma-Aldrich, Germany) is added to a final concentration of 20 micrograms/mL, and incubation continued for a further one hour. All steps from this point onward are performed at either 4° C. or on ice. The cells are centrifuged at 4500 rpm for 15 minutes to pellet and the supernatant discarded. The cells are then washed twice in 15 ml ice cold 0.5 M sucrose/10% glycerol (Sigma-Aldrich, Germany; SG) solution. Finally, the cells are resuspended in 1 mL SG and 100 microliters aliquots are immediately stored at −80° C. until electrotransformation as described below.

### 9. Electrotransformation & Transformant Selection

Electrotransformation is performed using freshly prepared competent cells as described above, with the following modifications: competent cells are defrosted on ice for 5 mins, 100 nanograms of plasmid is added and the solution is gently mixed. The mixture is transferred to a pre-chilled 2 mm electroporation cuvette (Cell Projects, U.K.) and electroporated under the following conditions: 1.75 kV/400 Ohm/25 microF. Nine hundred and fifty microliters of recovery broth (PDM broth with the addition of 20 mM MgCl₂ (Sigma-Aldrich, Germany)) is immediately added and the transformed cells are recovered at 42° C. for three hours. One hundred microliters of undiluted and, where appropriate, diluted transformed cells are plated on antibiotic selection plates (PDM agar containing 5 micrograms/mL chloramphenicol (Sigma-Aldrich, Germany); PDM-Cm5) and incubated ON at the appropriate temperature.

Following ON incubation of plates, 10 colonies per condition are re-streaked onto fresh PDM-Cm5 agar plates and incubated at 42° C. until individual colonies appeared. Colonies representing potential transformants are screened by colony PCR. In the case of transformants used in Example 1 primers DBC-23245 (SEQ ID NO: 7) and DBC-23246 (SEQ ID NO: 8) were used to check for presence of plasmid. One correct transformant per construct is selected and purified on PDM-Cm5 agar plates and further grown on 10% RSM-Cm5 and used as inoculum for further assays (described below). Transformants for each construct are listed in Table 1.

### 10. Plasmid Curing

In order to cure transformants from the introduced pNZ44 and pNZ44-derived plasmids, selected transformants are subjected to five ON passages at 42° C. in 10% RSM without the addition of chloramphenicol. Overnight cultures are then ten-fold serially diluted in ¼ strength Ringers solution (Merck, Germany), or a plate streak performed, and individual colonies are assessed for sensitivity to chloramphenicol by streaking on PDM-Cm5 agar plates. Streaked colonies which show no growth on PDM-Cm5 agar but growth on PDM agar are defined as cured transformants.

### 11. Acidification

ON cultures of S. thermophilus DS68802 and derived BIMs are used to inoculate 10 or 12% reconstituted skim milk (RSM, Darigold America) to measure acidification. Fermentation volumes are at least 30 mL of 10 or 12% RSM. Fermentation temperature is kept at 40 or 42° C. and acidification is measured using the CINAC system (Alliance Instruments) or suitable pH probes. Fermentations are stopped after 20 hours.

### Example 1

### Finding Anti-CRISPR Protein Expressing Bacteriophages

### Phage Genome Sequencing and Mining for Anti-CRISPR Protein Encoding Genes

Firstly, a virulent phage expressing anti-CRISPR protein is either recombineered to enable infection of a selected host, or an available virulent phage with anti-CRISPR protein encoding gene is selected. Currently, AcrIIA5 and AcrIIA6 are described in S. thermophilus phages (Hynes et al., 2018, Nature Communications, 9, 2919). In order to increase the occurrence of non-CRISPR BIMs one could selectively inactivate CRISPR immunity by selecting a test phage for BIM derivation which expresses an anti-CRISPR protein. A person skilled in the art can find anti-CRISPR protein expressing phages by sequencing S. thermophilus phage genomes by e.g. Illumina or Nanopore (Oxford) sequencing methods, and mining the genome sequences with BLAST searches to look for orthologs of known anti-CRISPR proteins, such as AcrIIA5 and AcrIIA6 (Hynes et al., 2018, Nature Communications, 9, 2919) or other anti-CRISPR protein gene sequences in the phage genomes. With a similar approach, in a screen of 254 S. thermophilus phage genomes, Hynes et al. (2018, Nature Communications, 9, 2919) found that 35% contained an acrIIA6 ortholog and 5.5% contained an acrIIA5 ortholog with at least 75% sequence identity over 75% of the gene length. W02018/197495 describes 233 potential anti-CRISPR protein encoding genes grouped in 84 families by in silico analysis using conserved neighbourhood analysis followed by restriction on predicted protein size and elimination of genes with known function.

### Verifying Anti-CRISPR Activity of Gene Sequences

Similar phage genome analysis can be conducted to select for phages for selection in BIM derivation with an anti-CRISPR ortholog in the genome. Three putative anti-CRISPR protein-encoding genes were found in the genome sequences of the DSM phage collection: acr100E-D1 (from φ100-E-D1A-L infecting S. thermophilus strain 100-E; SEQ ID NO: 2, 156 base pairs in length), acr200FD77 (from φ200-F-D77; SEQ ID NO: 3; 156 base pairs in length), and acr300F (from φ300F; SEQ ID NO: 4; 336 basepairs in length). The previously proven functional anti-CRISPR protein in S. thermophilus, the acrIIA5 gene sequence isolated from φD4276 (Hynes et al., 2017 Nature Microbiol, 2, 1374-1380; SEQ ID NO: 5; 423 base pairs in length) was included in the study. Table 2 shows the alignment of the three putative anti-CRISPR protein gene sequences and two confirmed anti-CRISPR proteins (acrIIA5 from ϕD4276 and acrIIA6 from ϕD1881). Table 3 shows a similar table but then the identity between protein sequences.

Similar as described by Hynes et al., 2017, anti-CRISPR activity of the gene sequences was verified by expression of the gene sequences in CRISPR-immunized strains and determining the virulence of phages to which CRISPR immunity has been acquired. DS75685 and DS75686 are both BIMs derived from strain 100-E. Sequencing analysis of the CRISPR loci in both strains showed addition of two spacers in CRISPR1 and three spacers in CRISPR3 for DS75685 matching to sequences in all four phages (ϕ100E-D1A-L, ϕ100E-D2A-L, ϕ100E-D3A-L and ϕ100E-D4A-L, and addition of one spacer in CRISPR1 and four spacers in CRISPR3 for DS75686 with spacer sequences also matching to all four phages.

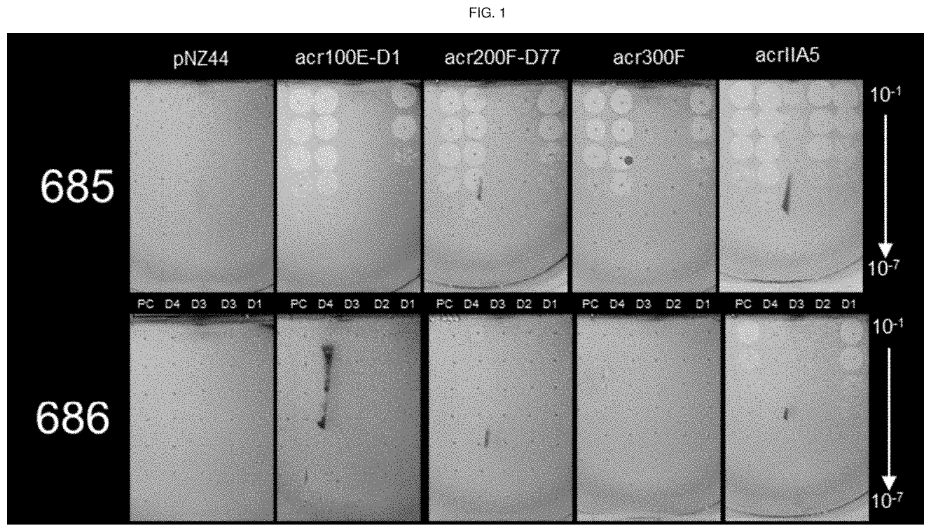

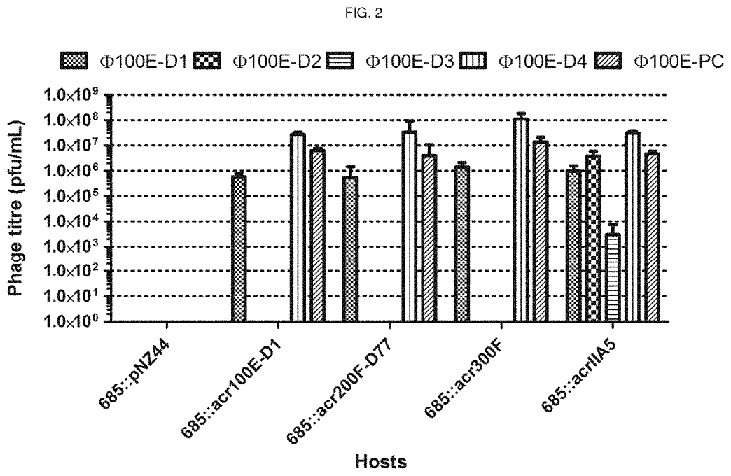

Each anti-CRISPR protein-encoding gene was cloned into high copy plasmid pNZ44. DS75685 and DS75686 were transformed with each anti-CRISPR plasmid and empty vector. PCR-verified transformants of each anti-CRISPR plasmid and empty vector control strains (listed in Table 1) were assayed by double agar overlay assay in triplicate and the phage infection titres for the four individual phages infecting parent strain 100-E and a phage cocktail of all four (PC) were recorded for each tested strain. FIG. 1 displays a representative result of the double agar spot assay. DS75685 transformed with empty plasmid (685::pNZ44) was unsensitive to all four individual phages as well as the phage cocktail, whereas the DS75685 transformants expressing putative anti-CRISPR proteins regained sensitivity to at least two of the four phages, with 685::acrIIA5 becoming sensitive to all four phages. The results of the triplicate experiments showed this profile of the transformants was consistent across experiments (FIG. 2). This indicated that CRISPR-immunity was mostly responsible for the phage-insensitivity of DS75685. DS75686 transformed with empty plasmid (686::pNZ44) was unsensitive to all four individual phages and the phage cocktail and except for 686::acrIIA5 no sensitivity was observed for the anti-CRISPR protein transformants. Regained sensitivity of 686::acrIIA5 was only observed in the case of infection with ϕ100E-D4A-L, and the phage cocktail of which ϕ100E-D4A-L was also part. The results for DS75686 transformants indicated that insensitivity of DS65686 was partly contributed by CRISPR immunity but also other mechanisms. Possibly, other mutations resulted in phage insensitivity by using the mutagenesis BIM derivation approach.

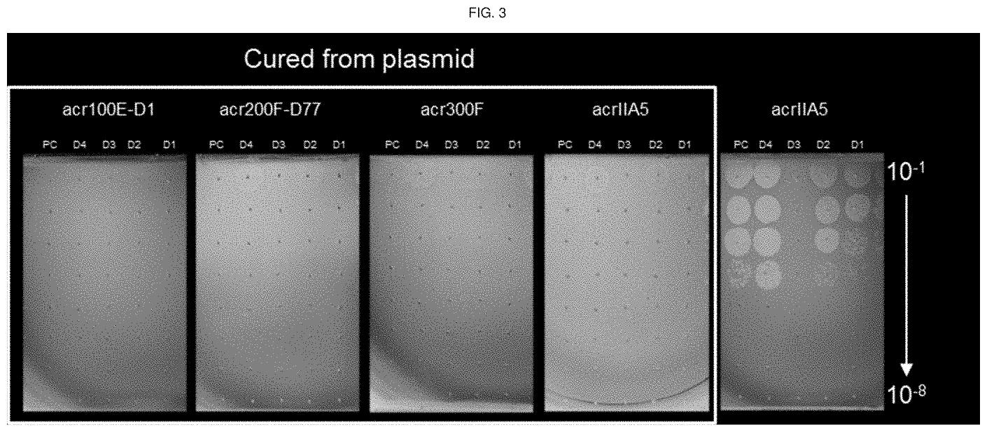

To show that the regained sensitivity was the result of the expression of the anti-CRISPR proteins DS75685 transformants were cured from their plasmid by serial cultivation on 10% RSM without chloramphenicol in the medium. The resulting double agar spot assay showed that the cured transformants became insensitive to all four phages again indicating that the previous sensitivity was caused by the expression of anti-CRISPR proteins from the introduced plasmids. (FIG. 3).

In summary, all anti-CRISPR proteins tested displayed functionality of inactivating CRISPR immunity (especially in DS75685), of which AcrIIA5 displayed the broadest effect for all four phages against 100-E. The phages these sequences originate from can thus be used for BIM derivation for the respective strains they are able to infect.

### Example 2—BIM derivation conditions of DS68802

Another example of using phage genome sequencing and genome mining with known anti-CRISPR protein encoding genes was found in the DSM phage collection in which three phages against S. thermophilus DS68802 were found (ϕ643-01, ϕ643-03, ϕ643-11) of which one (ϕ643-11) encoded a gene sequence 95% identical to the gene sequence of AcrIIA5 (described in Hynes et al., 2017 Nature Microbiol, 2, 1374-1380).

BIM Derivation Conditions with DS68802

A set of different BIM derivation conditions is applied to derive BIMs from S. thermophilus DS68802 (as depicted in Table below). Optionally, mutagenesis is applied on DS68802 with a suitable mutagen (e.g. NTG, NQO, UV, irradiation). NTG mutagenesis has been described in Material and methods, as example. As first round selecting phage(s), ϕ643-01, ϕ643-03, or ϕ643-11 (encoding anti-CRISPR protein ortholog of acrIIA5) are each applied as selection phage in the double agar method. For condition 1 to 3, if available >90 colonies are isolated and stored for further characterization. For condition 4 to 6, all surviving colonies after phage selection are pooled and subjected to 20 cultivation cycles on 10% RSM exposed with a specific phage dependent on the condition, as depicted in the table below. After the last cycle, if available >90 colonies are isolated and stored for further characterization as explained further below.

TABLE 4
BIM derivation protocol conditions
for BIM derivation of DS68802

| | | | Challenging |
|---|---|---|---|
| | | Additional cultivation | phage during |
| | Selection | cycles with | 20 cycles on |
| Condition | phage | phage challenge? | 10% RSM |
| 1 | ϕ643-01 | No | n.a. |
| 2 | ϕ643-03 | No | n.a. |
| 3 | ϕ643-11 (ACR) | No | n.a. |
| 4 | ϕ643-01 | Yes | ϕ643-01 |
| 5 | ϕ643-03 | Yes | ϕ643-03 |
| 6 | ϕ643-11 (ACR) | Yes | ϕ643-11 (ACR) |

ACR = anti-CRISPR protein encoding gene present in phage

### Heap-Lawrence Assays

The potential BIMs per condition are grown in 10% milk without phage exposition to verify their growth properties in 10% RSM. If available, ≥96 colonies per condition are to be screened. For

BIMs derived from DS68802 selected according to conditions 4, 5, and 6, a higher percentage of sufficiently growing colonies on milk is expected, than for BIMs derived from DS68802 selected according to conditions 1, 2 and 3.

The potential BIMs which are still able to grow on milk, selected per condition are subjected to a first Heap-Lawrence assay for 3 cycles using as starting phage(s) the phage(s) used in primary selection and/or exposition during cultivation cycles. For BIMs derived from DS68802 selected on the anti-CRISPR protein encoding gene-bearing phage ϕ643-11 (conditions 3 and 6), a higher percentage of surviving colonies is expected after these initial three cycles of Heap-Lawrence, than for BIMs derived from DS68802 selected on ϕ643-01 (conditions 1 and 4) or ϕ643-03 (conditions 2 and 5).

Subsequently, with the surviving BIMs after 3 cycles, an infinite Heap-Lawrence assay is executed with a starting phage cocktail of all three phages ϕ643-01, ϕ643-03 and ϕ643-11. In this way, the amount of acidification cycles under stringent phage pressure can be determined for each BIM. On average, non-CRISPR BIMs are expected to survive most cycles in the infinity Heap-Lawrence assay. Therefore, it is expected that the average number of cycles survived by BIMs derived from DS68802 selected on ϕ643-11 (conditions 3 and 6) is higher than that of DS68802 selected on ϕ643-01 (conditions 1 and 4) or ϕ643-03 (conditions 2 and 5).

### Screening of CRISPR1 and CRISPR3 Loci for Spacer Addition

If available, ten to twenty BIMs of the ≥90 potential BIMs per condition are screened for spacer addition on the CRISPR1 and CRISPR3 loci with either PCR with suitable primers to determine spacer acquisition based on amplicon size (Hynes et al., 2017, Nature Microbiol, 2, 1374-1380), or with Sanger sequencing of the entire CRISPR locus. The percentage of the total number of BIMs with spacer additions is determined between conditions. Potential BIMs derived from DS68802 selected on ϕ643-11 (conditions 3 and 6), are expected to have a lower percentage of colonies with spacer additions than the BIMs derived from DS68802 selected on ϕ643-01 (conditions 1 and 4) or ϕ643-03 (conditions 2 and 5) when comparing the same mutagenesis conditions across phage selections.

### Phage Sensitivity Overlay Assay

If available, for ten selected BIMs per condition phage sensitivity assays are to be conducted as described above in Material and Methods.

Each BIM and parent strain DS68802, are grown in top agar and 10 ul of 10⁻¹ to 10⁻⁸ dilutions of phage stock are spotted as described above, and after ON incubation 24 hrs at 42° C., EOPs are determined for each host-phage combination. Table 3 describes the average EOPs measured for each condition/phage. On average, the EOP for BIMs derived from DS68802 selected on ϕ643-11 (condition 3 and 6) is lower than the EOP for BIMs derived from DS68802 selected on ϕ643-01 (condition 1 and 4) or ϕ643-03 (condition 2 and 5) for more than one phage tested (out of three phages in the case of DS68802).

Acidification of BIMs Derived with BIM Derivation Conditions

From the assays above, the highest percentages of non-CRISPR BIMs with broad spectrum of immunization are expected from BIM derivation conditions 3 and 6. Therefore, BIMs from these conditions were further assessed for application performance. For the selection of BIMs to be used in a commercial starter culture for dairy applications, maintained acidification performance is an essential qualification criterium. Criteria for acidification performance for BIMs are TTR of pH 5.2 and pH after 5 hours of fermentation in CINAC at 42° C. compared to the parent strains DS68802. Ten BIMs for each condition derived with BIM derivation protocol conditions 3 and 6 are evaluated.

On average, the TTR for pH 5.2 is shorter for BIMs derived from DS68802 selected on ϕ643-11 followed by cultivation cycles on milk challenged with ϕ643-11 (condition 6) than for BIMs derived from DS68802 selected on ϕ643-11 without cultivation cycles on milk challenged with ϕ643-11 (condition 3).

Furthermore, on average, the pH after 5 hours of fermentation of milk is lower for BIMs derived from DS68802 selected on ϕ643-11 followed by cultivation cycles on milk challenged with ϕ643-11 (condition 6) than for BIMs derived from DS68802 selected on ϕ643-11 without cultivation cycles on milk challenged with ϕ643-11 (condition 3).

Results above exemplify that BIMs derived under condition 6 are enriched in a higher percentage for non-CRISPR BIMs with a maintained acidification performance.

### Example 3: DS68802 BIM Derivation and Characterization after Four Cycles of Heap-Lawrence

BIM derivation was conducted essentially as described in the Methods section (section 3). For this example, DS68802 cells were processed without mutagenesis. For the double agar selection plates, a top agar was prepared by supplementing top agar (1:2 broth:agar) with 5 mM CaCl₂ and phage lysate (10⁷ pfu/mL for 1:10 ratio and 10⁶ pfu/mL for 1:1 ratio). Four mL of the phage containing top agar was poured on each plate. Five plates were prepared for each of the three phage selection conditions:

1) ϕ643-01 in a cell : phage ratio of 1:10

2) ϕ643-11 in a cell : phage ratio of 1:10

3) ϕ643-11 in a cell : phage ratio of 1:1

Freshly pre-cultured DS68802 cells were diluted to a cell concentration of 10⁶ cfu/mL and 100 μl aliquots were plated on the phage selection plates resulting in the above-mentioned phage:cell ratio's. Plates were incubated at incubation temperature of 42° C. for 3-4 days until visible colonies appeared. For selection conditions 1 and 2, 74 and 96 single colonies (BIMs) were picked, respectively, and grown overnight in 300 microliters of 10% RSM. Twenty microliters of an overnight culture of the BIMs was transferred to 180 microliters of 10% RSM supplemented with 0.6% bromecresol purple (BCP; Sigma-Aldrich) in a microtiter plate. Plates were incubated overnight at 42° C. and the culture was resuspended with 180 microliters of 10% RSM supplemented with 0.6% bromecresol purple. The resuspended culture was subsequently diluted ten times by adding 15 microliters resuspended culture to 135 microliters of 10% RSM supplemented with 0.6% bromocresol purple into new 96-deepwell plates. By repeating the previous, at least 6 replicate plates were made which were stored at −80° C. for the Heap Lawrence assay. For condition 3, the entire top agar overlay was scrapped off with sterile spatula and used to inoculate 5 mL of 10% RSM medium which was then incubated overnight at 42° C. Finally, 1% inoculum was then used to inoculate 5 mL of 10% RSM and passed 20 times through fresh 10% RSM containing 4)643-11 at a MOI of 10. After the last cycle, cultivated cells were plated out on PDM-agar plates and twelve colonies were picked and passed through the master plate preparation as described above for conditions 1 and 2.

To verify that the twelve selected BIMs were related to the parent strain DS68802 they were derived from, the twelve BIMs were freshly cultured on PDM-agar and colony material was suspended in 50 μl milli-Q and 1 μl was used as template in a 25 μl reaction. With this material as template, a so called “Random Amplification of Polymorphic DNA” (RAPD) colony PCR was performed using illustra PureTaq Ready-to-go PCR beads (GE Healthcare, 27-9559-01) according to suppliers' instructions. The following primer and PCR conditions were used:

GTG5:
5′ gtggtggtggtggtg 3′
PCR program GTG5:

1 step: 7′ 95° C.
30 cycles of: 30″ 90° C., 1′ 40° C., 8′ 65° C.
1 step: 10′ 65° C.
Hold at 12° C.

Analysis of the PCR product by gel electrophoresis on 2% agarose generated a unique strain specific random pattern of PCR fragments. This analysis allowed comparing the parent strain and BIMs to draw conclusions on their relatedness (to make sure they are indeed related). Any difference in RAPD PCR pattern would indicate a potential switch or contamination in the derivation. For the twelve BIMs, no differences in patterns were discerned confirming their relatedness to parent strain DS68802.

The stored BIMs in the master plates were subjected to the Heap-Lawrence assay which was performed similarly as described in the Methods section (section 4). For each Heap-Lawrence cycle, one of the stock plates was taken from the −80° C. freezer and was incubated overnight at 42° C. In microtiter plates containing 350 microliters of 10% RSM+0.6% BCP, 50 microliters of a 5-times diluted overnight culture was inoculated, along with 50 microliters of 10-fold diluted bacteriophage solution of either a single phage isolate or a mixture of phages. A control was included, where 350 microliters of 10% RSM+0.6% BCP was inoculated with 50 microliter 5-fold diluted overnight culture to 300 microliter 10% RSM+0.6% BCP without the presence of the bacteriophage. Following incubation for five hours at 42° C., the plates were measured using the Delvo®Scan. With the Delvo®Scan software the pH-dependent coloration of BCP was quantified and resulted in a Z-value based on the RGB of the measured colour. A high Z-value (between −1 and −10) indicates that the BIM has failed to clot and acidify the media and, thus, is susceptible to the bacteriophage in the culture, whereas a low Z-value (more negative than −15) indicates that the BIM has survived the bacteriophage predation and has successfully acidified the culture and clotted the milk. Between −10 and −15 are intermediate cases. After each acidification (Heap-Lawrence) cycle, content from each plate containing phages was pooled in a 50 ml Greiner tube. Optionally, a few drops of 1 M lactic acid solution were added to induce clotting of milk. Tubes were centrifuged at 4,000 g for 5 minutes followed by filter-sterilization (0.45 μm pore size) of the suspension. Finally, the pH was adjusted to 7 using 0.8 M KOH. These suspensions contained the evolved bacteriophages against the isolated BIMs and were then used in combination with the single bacteriophage isolate suspension for the next cycle of Heap Lawrence (added 1:1 with original phage). Four rounds of Heap Lawrence were performed in this manner. Table 5 below depicts the phage lysate as selection used for each Heap-Lawrence cycle and for each single phage isolate selection condition of the respective BIMs:

TABLE 5

| Condition | Cycle 1 | Cycle 2 | Cycle 3 | Cycle 4 |
|---|---|---|---|---|
| 1 | ϕ643-01 | ϕ643-01 + HL1 | ϕ643-01 + HL2 | ϕ643-01 + HL3 + |
| | | | | ϕ643-11 |
| 2 | ϕ643-11 | ϕ643-11 + HL1 | ϕ643-11 + HL2 | ϕ643-11 + HL3 + |
| | | | | ϕ643-01 |
| 3 | ϕ643-11 | ϕ643-11 + HL1 | ϕ643-11 + HL2 | ϕ643-11 + HL3 + |
| | | | | ϕ643-01 |

HL = pooled phage lysate from Heap-Lawrence;
number indicates the cycle of Heap-Lawrence the pooled phage lysate originated from.

After the fourth cycle of Heap Lawrence, the distributions of Z-values measured after 5 hours with phage challenge for BIMs per condition are listed in Table 6 below.

TABLE 6

| Condition | Z-value > −10 | −10 > Z-value > −15 | Z-value < −15 |
|---|---|---|---|
| 1 | 54% | 18% | 28% |
| 2 | 41% | 9% | 50% |
| 3 | 0% | 0% | 100% |

BIMs which survive four cycles of Heap-Lawrence show a high degree of stability of their acquired phage insensitivity. Also, the incidence increases of mutations responsible for the insensitivity are of a non-CRISPR nature, since CRISPR mutants tend to survive a lower amount of cycles in Heap-Lawrence. Table 6 indicates that the incidence of survivors which are able to fully clot milk (Z-value <−15) is higher when using ϕ643-11 (condition 2 and 3) as selection condition than when using ϕ643-01 as selection condition. The genome of ϕ643-11 encodes an anti-CRISPR gene, whereas ϕ643-01 does not. Surprisingly, when 20 cycles of co-cultivation with ϕ643-11 were conducted in the BIM derivation protocol, all isolated BIMs survived four cycles of Heap-Lawrence and showed a high degree of acidification after five hours with phage predation (condition 3).

This result indicates that a 100% success rate was achieved for derivation of stable DS68802 BIMs with a high acidification rate, when selected on the anti-CRISPR gene-bearing phage ϕ643-11 followed by twenty cycles of co-cultivating mutants with ϕ643-11. When applying only selection on ϕ643-11 without the co-cultivation cycles (condition 2) the success rate in finding suitable stable DS68802 BIMs was halved.

### Example 4: DS68802 BIM Derivation when Applying Mutagenesis and Characterization after Four Cycles of Heap-Lawrence

BIM derivation was conducted essentially as described in the Methods section (section 3). For this example, DS68802 cells were processed through the protocol with mutagenesis using NTG as a mutagen. In classical strain improvement, such as strain hardening against phages, mutagenesis is applied routinely to increase the diversity of mutants. The downside of applying mutagenesis is that many strains acquire mutations that do not improve the performance in the intended application. Therefore, a large set of mutants need to be screened with varying success rates. By applying selection of BIMS on an ant-CRISPR gene-bearing phage combined with co-cultivation cycles with the same phage, it is hypothesized that the success rate in finding suitable BIMs with a stable (non-CRISPR-mediated) phage insensitivity profile and maintaining acidification rates similar to the parent in a population of mutagenized cells is increased. Thereby, hopefully finding rare mutants with an even more stable phage insensitivity profile which does not compromise the acidification rate.

DS68802 cells were mutagenized essentially according to Section 3 of the Methods section. For breaking up cell chains, DS68802 cells were sonicated 7×30 seconds with the Soniprep 150, MSE) set at 30 seconds with amplitude 10 μm and 30 seconds pause between each cycle. During sonication the culture was constantly cooled in ice water. For mutagenesis, 0.0125 micrograms/mL NTG was applied.

For the double agar selection plates, a top agar was prepared by supplementing top agar (1:2 broth:agar) with 5 mM CaCl₂ and phage lysate (10⁷ pfu/mL for 1:10 ratio and 10⁶ pfu/mL for 1:1 ratio). Four mL of the phage containing top agar was poured on each plate. Five plates were prepared for each of the three phage selection conditions:
1) ϕ643-01 in a cell : phage ratio of 1:10
2) ϕ643-11 in a cell : phage ratio of 1:10
3) ϕ643-11 in a cell : phage ratio of 1:1

Mutagenized DS68802 cells were diluted to a cell concentration of 10⁶ cfu/mL and 100 μl aliquots were plated on the phage selection plates resulting in the above-mentioned phage:cell ratio's. Plates were incubated at incubation temperature of 42° C. for 3-4 days until visible colonies appeared. For both selection conditions 1 and 2, 96 single colonies (BIMs) were picked. Stock plates of these BIMs were prepared as described in Example 3. For condition 3, the entire top agar overlay was scraped off with sterile spatula and used to inoculate 5 mL of 10% RSM medium which was then incubated ON at 42° C. Finally, 1% inoculum was then used to inoculate 5 mL of 10% RSM and passed 20 times through fresh 10% RSM containing ϕ643-11 at a MOI of 10. After the last cycle, cultivated cells were plated out on PDM-agar plates and twelve colonies were picked and passed through the stock plate preparation as described in Example 3.

To verify that the twelve selected BIMs were related to the parent strain DS68802 they were derived from, the twelve BIMs were freshly cultured on PDM-agar and colony material was suspended in 50 μl milli-Q and 1 μl was used as template in a 25 μl PCR reaction. With this material as template, a so called “Random Amplification of Polymorphic DNA” (RAPD) colony PCR was performed as described in Example 3.

For the twelve BIMs no differences in patterns were discerned confirming their relatedness to parent strain DS68802.

The stored BIMs (for condition 1, 2 and 3) from the −80 stock plates were subjected to the Heap-Lawrence assay which was exactly performed as described in Example 3. Table 7 below depicts the phage lysate as selection used for each Heap-Lawrence cycle and for each original phage selection condition of the respective BIMs:

TABLE 7

| Condition | Cycle 1 | Cycle 2 | Cycle 3 | Cycle 4 |
|---|---|---|---|---|
| 1 | ϕ643-01 | ϕ643-01 + HL1 | ϕ643-01 + HL2 | ϕ643-01 + HL3 + |
| | | | | ϕ643-11 |
| 2 | ϕ643-11 | ϕ643-11 + HL1 | ϕ643-11 + HL2 | ϕ643-11 + HL3 + |
| | | | | ϕ643-01 |
| 3 | ϕ643-11 | ϕ643-11 + HL1 | ϕ643-11 + HL2 | ϕ643-11 + HL3 + |
| | | | | ϕ643-01 |

HL = pooled phage lysate from Heap-Lawrence;
number indicates the cycle of Heap-Lawrence the pooled phage lysate originated from.

After the fourth cycle of Heap Lawrence the distributions of Z-values measured after 5 hours with phage challenge for BIMs per condition are listed in Table 8 below.

TABLE 8

| Condition | Z-value > −10 | −10 > Z-value > −15 | Z-value < −15 |
|---|---|---|---|
| 1 | 73% | 9% | 18% |
| 2 | 60% | 4% | 36% |
| 3 | 0% | 8% | 92% |

Similar as in Example 3, Table 8 indicates that the incidence of survivors which are able to fully clot milk (Z-value <−15) is higher when using as selection condition ϕ643-11 (condition 2 and 3) than using ϕ643-01. However, the percentage of BIMs (36%) showing fast acidification in the fourth cycle was lower than for Example 3, where 50% showed the desired performance in the fourth cycle of Heap-Lawrence. This reflects that with applying mutagenesis the success rate is lowered in finding suitable, stably phage-insensitive, fast acidifying candidates in a set of nearly 100 BIMs. Thereby, demanding a larger screening effort. However, when 20 cycles of co-cultivation with ϕ643-11 were conducted in the BIM derivation protocol, then 92% (11 out of 12) isolated BIMs survived four cycles of Heap-Lawrence and showed a high degree of acidification after five hours with phage predation (condition 3).

This result indicates that a 92% success rate was achieved for derivation of stable DS68802 BIMs with a high acidification rate, when selected on the anti-CRISPR gene-bearing phage ϕ643-11 followed by twenty cycles of co-cultivating mutants with ϕ643-11. When applying only selection on ϕ643-11 without the co-cultivation cycles the success rate in finding suitable stable DS68802 BIMs was only about a third of that.

1. A method for obtaining a non-CRISPR bacteriophage-insensitive mutant (BIM) from a lactic acid bacterium parent strain comprising
f. exposing said parent strain to at least one virulent bacteriophage which expresses a gene encoding an anti-CRISPR protein
g. growing surviving lactic acid bacteria for at least one cycle in milk in the presence of said at least one bacteriophage which expresses a gene encoding an anti-CRISPR protein
h. isolating BIMs resulting from b
i. characterizing at least one isolated BIM by
i. exposing said isolated BIM to at least two different bacteriophages to determine the spectrum of bacteriophage insensitivity, and
ii. testing the stability of the acquired bacteriophage resistance of said at least one isolated BIM, and
iii. determining the acidification profile of said isolated BIM in milk
j. and selecting a BIM which
i. when compared to the parent strain is insensitive to more bacteriophages as determined in d (i), and
ii. when compared to the parent strain has an improved stability of the acquired bacteriophage resistance as determined in d(ii), and
iii. has an acidification profile, as determined in d(iii), in milk which is at least comparable to or improved when compared to said parent strain.

2. The method according to claim 1 wherein d ii. comprises
A. growing an isolated BIM in milk in the presence of a bacteriophage
B. obtaining bacteriophages from the resulting milk of A, and
C. growing said isolated BIM in milk in the presence of newly added bacteriophage and the bacteriophages obtained in B,
D. repeating obtaining bacteriophages from the resulting milk and growing of said isolated BIM in milk in the presence of newly added bacteriophage and the obtained bacteriophages for at least a total of 3 cycles, to determine the number of cycles of survival of an isolated BIM as a measure for stability of insensitivity in batch cultures with evolving bacteriophage present.

3. The method according to claim 1, wherein said lactic acid bacterium parent strain is a Streptococcus thermophilus parent strain.

4. The method according to claim 1, wherein said lactic acid bacterium parent strain is mutagenized.

5. The method according to claim 1, wherein b comprises at least 2, 3, 4, 5, 6, 7, 8, 9 or 10, optionally at least 15, optionally at least 20, optionally at least 25 cycles.

6. The method according to claim 2, wherein the amount of cycles in d(ii)(D) comprises at least 5 or 10, optionally at least 15, optionally at least 20, optionally at least 25 cycles.

7. The method according to claim 1, wherein a comprises exposing said parent strain to at least two different virulent bacteriophages and wherein at least one of said two different bacteriophages is a virulent bacteriophage which expresses a gene encoding an anti-CRISPR protein.

8. The method according to claim 1, further comprising adding a cryoprotectant to the obtained bacteriophage insensitive mutant.

9. The method according to claim 1, further comprising freeze drying or freezing the bacteriophage insensitive mutant.

10. A starter culture composition suitable for inoculation of a medium to be fermented on an industrial scale comprising a bacteriophage insensitive mutant as obtained by the method of claim 1.

11. The starter culture composition according to claim 10, wherein the starter culture composition is frozen, freeze dried or in liquid form.

12. A process for production of a dairy product optionally a fermented milk product or cheese product comprising using one or more bacteriophage-insensitive mutants as obtained by the method of claim 1 or comprising using a starter culture composition comprising said one or more of said mutants.

13. A product comprising a bacteriophage-insensitive mutant of the lactic acid bacterium Streptococcus thermophilus parent strain as obtained by the method of claim 1 a starter culture composition comprising said mutant adapted for use in a process for the production of a dairy product, optionally a fermented milk or cheese.

14. The product according to claim 13 wherein said lactic acid bacterium parent strain is a Streptococcus thermophilus parent strain.
